(19) United States
(12) Patent Application Publication
(10) Pub. No.: US 2021/0388029 A1
(43) Pub. Date: Dec. 16, 2021

(54) CYTOPLASMIC TRANSDUCTION PEPTIDE AND INTRACELLULAR MESSENGER COMPRISING SAME

(71) Applicant: AVIXGEN INC, Seoul (KR)

(72) Inventors: Yi Yong BAEK, Goyang-si Gyeonggi-do (KR); Min Jung KIM, Seoul (KR)

(73) Assignee: AVIXGEN INC, Seoul (KR)

(21) Appl. No.: 17/303,250

(22) Filed: May 25, 2021

Related U.S. Application Data
(63) Continuation of application No. 16/469,632, filed on Jun. 13, 2019.

(30) Foreign Application Priority Data
Dec. 16, 2016 (KR) 10-2016-0172548
Dec. 15, 2017 (KR) 10-2017-0173642

(51) Int. Cl. C07K 14/005; A61K 47/62

(52) U.S. Cl. CPC C07K 14/005; C12N 2740/16071; C12N 2740/16022; A61K 47/62

(57) ABSTRACT

The present invention relates to a cell membrane penetrating peptide and an intracellular delivery carrier including the same. The intracellular delivery carrier of the present invention has an advantage of efficiently transferring substances into cells even at a low concentration thereof compared with the existing cell membrane penetrating peptide derived from the virus.

### CROSS-REFERENCE TO RELATED APPLICATIONS

This application is a continuation of U.S. application Ser. No. 16/469,632, which is a 371 of PCT/KR2017/014897, filed on Dec. 15, 2017 which claims the benefit of Korean Patent Application No. 10-2016-0172548, filed Dec. 16, 2016 and Korean Patent Application No. 10-2017-0173642, filed Dec. 15, 2017, the contents of each of which are incorporated herein by reference.

### REFERENCE TO A SEQUENCE LISTING SUBMITTED ELECTRONICALLY VIA EFS-WEB

The content of the electronically submitted sequence listing, file name: 4061-1006-C_SubSeqList20210903.txt; size: 9,000 bytes; and date of creation: Sep. 3, 2021, filed herewith, is incorporated herein by reference in its entirety.

### TECHNICAL FIELD

The present invention relates to a cell membrane penetrating peptide and an intracellular delivery carrier including the same.

### BACKGROUND OF THE INVENTION

The nucleocapsid protein (hereinafter referred to as ‘NC’) of the human immunodeficiency virus (HIV) plays a functional role in the viral life cycle as well as the structural role for viral growth. The functions are as follows. First, NC peptides are involved in the genomic encapsulation of viruses. This function results from two zinc finger domains consisting of unique CCHC motifs. The domains are known to be highly conserved in all retroviruses and are essential for HIV RNA packaging and infectious virus production. Second, NC peptides are known to promote tRNA primer annealing and strand transfer during viral reverse transcription (RT), suggesting that NC peptides play an important role in viral replication. Third, NC peptides have the nucleic acid chaperone activity necessary for the viral life cycle. Recently, it has been reported that NC peptides play a predetermined role even when the viral DNA is inserted into the host cell chromosome.

Meanwhile, cell penetrating peptides (CPP) are cell membrane penetrating peptides composed of about 10-30 short peptides. Most of them are derived from protein-transduction domain or membrane-translocating sequence. Unlike the general intracellular entry pathway of foreign substances, CPP is known to be capable of transferring DNA and proteins that are known to be unable to pass through cell membranes into cells without damaging the cell membrane. The best-known peptides for CPP are the Tat peptides, which are derived from Human Immunodeficiency Virus (HIV) and are currently being used in a variety of applications, such as cell therapy and diagnostic reagents. In addition, Penetratin, derived from the DNA-binding domain of a homeodomain transcription factor, is also being used to deliver proteins useful to the human body to the skin. The transportan is a peptide made by fusing some of the peptides isolated from the venom of the wasp called mastoparan with a neuropeptide called galanin and is used as a cell death-inducing peptide by binding with a peptide inducing cell death.

The present inventors have confirmed that NC peptides have cell membrane penetration activity while studying the physiological activity of HIV NC peptides so that NC peptides can be used as a drug delivery carrier capable of delivering intracellular substances, thereby completing the present invention.

### SUMMARY OF THE INVENTION

### Technical Problem

An object of the present invention is to provide a cell penetrating peptide including an amino acid sequence represented by the following Formula I:

[Formula I]
(SEQ ID NO: 16)
Cys-Xaa₁-Xaa₂-Cys-Xaa₃-Xaa₄-Xaa₅-Gly-His-Xaa₆-Xaa₇-
Xaa₈-Xaa₉-Cys

in which Xaa₁ is an amino acid selected from the group consisting of Ala, Val, Ile, Leu, Met, Phe, Tyr, Trp, Ser, Thr, Asn and Gln,

in which Xaa₂ is an amino acid selected from the group consisting of Ala, Val, Ile, Leu, Met, Phe, Tyr, Trp, Arg, His, Lys, Asn, Ser, Thr and Gln,

in which Xaa₃ and Xaa₄ are individually an amino acid selected from the group consisting of Asp, Glu, Arg, His, Lys, Ser, Thr, Asn, Gln and Gly,

in which Xaa₅ is an amino acid selected from the group consisting of Asp, Glu, Arg, His, Lys, Ala, Val, Ile, Leu, Met, Phe, Tyr, Trp and Pro,

in which Xaa₆ and Xaa₇ are individually an amino acid selected from the group consisting of Ser, Thr, Asn, Gln, Ala, Val, Ile, Leu, Met, Phe, Tyr and Trp,

in which Xaa₈ is an amino acid of Lys, Ala or Arg, and

in which Xaa₉ is an amino acid selected from the group consisting of Asp, Glu, Ser, Thr, Asn and Gln.

Another object of the present invention is to provide an intracellular delivery carrier in which a cargo of an object to be delivered into a cell is conjugated with the end of the cell penetrating peptide.

Still another object of the present invention is to provide a method for delivering a cargo of an object to be delivered into a cell, the method including contacting the intracellular delivery carrier with a cell.

Yet another object of the present invention is to provide an intracellular delivery carrier in which a cargo of an object to be delivered into a cell is conjugated with the end of an NC peptide of a retrovirus.

Yet another object of the present invention is to provide a method for delivering a cargo of an object to be delivered into a cell, the method including contacting an intracellular delivery carrier in which a cargo of an object to be delivered into a cell is conjugated with the end of an NC peptide of retrovirus with a cell.

### Technical Solution

In order to achieve the objects, an aspect of the present invention provides a cell penetrating peptide including an amino acid sequence represented by the following Formula I:

[Formula I]
(SEQ ID NO: 16)
Cys-Xaa₁-Xaa₂-Cys-Xaa₃-Xaa₄-Xaa₅-Gly-His-Xaa₆-Xaa₇-
Xaa₈-Xaa₉-Cys

in which Xaa₁ is an amino acid selected from the group consisting of Ala, Val, Ile, Leu, Met, Phe, Tyr, Trp, Ser, Thr, Asn and Gln,

in which Xaa₂ is an amino acid selected from the group consisting of Ala, Val, Ile, Leu, Met, Phe, Tyr, Trp, Arg, His, Lys, Asn, Ser, Thr and Gln,

in which Xaa₃ and Xaa₄ are individually an amino acid selected from the group consisting of Asp, Glu, Arg, His, Lys, Ser, Thr, Asn, Gln and Gly,

in which Xaa₅ is an amino acid selected from the group consisting of Asp, Glu, Arg, His, Lys, Ala, Val, Ile, Leu, Met, Phe, Tyr, Trp and Pro,

in which Xaa₆ and Xaa₇ are individually an amino acid selected from the group consisting of Ser, Thr, Asn, Gln, Ala, Val, Ile, Leu, Met, Phe, Tyr and Trp,

in which Xaa₈ is an amino acid of Lys, Ala or Arg, and

in which Xaa₉ is an amino acid selected from the group consisting of Asp, Glu, Ser, Thr, Asn and Gln.

Another aspect of the present invention provides an intracellular delivery carrier in which a cargo of an object to be delivered into a cell is conjugated with the end of the cell penetrating peptide.

As used herein, the term “intracellular delivery carrier” refers to a carrier capable of penetrating through the cell membrane and penetrating into the tissue.

As used herein, the term “peptide” or “polypeptide” refers to a linear molecule formed by linking amino acid residues together through peptide bonds and includes 4-70 amino acid residues, preferably 4-40 amino acid residues, more preferably 4-30 amino acid residues, and most preferably 4-20 amino acid residues.

The polypeptide represented by the above Formula I refers to the consensus sequence (Cys-Xaa-Xaa-Cys-Xaa-Xaa-Xaa-Xaa-His-Xaa-Xaa-Xaa-Xaa-Cys, SEQ ID NO:24) of the zinc finger domain present in the NC peptide of various retroviruses (for example, human immunodeficiency virus (HIV), murine leukemia virus (MLU), simian immunodeficiency virus (SIV), rous sarcoma virus (RSV), Feline immunodeficiency virus (FIV), and equine immunodeficiency virus (EIV)). The present inventors have confirmed through experiments that the polypeptides including the above sequences are excellent in cell membrane penetration activity and that they can be used as a drug delivery system capable of delivering intracellular substances.

According to an embodiment of the present invention, the Xaa₁ is an amino acid selected from the group consisting of Ala, Val, Ile, Leu, Met, Phe, Tyr and Trp as a hydrophobic amino acid and Ser, Thr, Asn and Gln as a polar amino acid. More preferably, the Xaa₁ is an amino acid selected from the group consisting of Phe, Trp, Tyr, Ala and Gln.

According to an embodiment, the Xaa₂ is an amino acid selected from the group consisting of Ala, Val, Ile, Leu, Met, Phe, Tyr and Trp as a hydrophobic amino acid, Arg, His and Lys as a basic amino acid and Ser, Thr, Asn and Gln as a polar amino acid. More preferably, the Xaa₂ is an amino acid selected from the group consisting of Asn, Thr, Lys, Leu and Tyr.

Further, according to an embodiment, the Xaa₃ and Xaa₄ are individually an amino acid selected from the group consisting of Asp, Glu, Arg, His, Lys, Ser, Thr, Asn, Gln and Gly as a hydrophilic amino acid. More preferably, the Xaa₃ is an amino acid selected from the group consisting of Gly, Asp and Lys. More preferably, the Xaa₄ is an amino acid selected from the group consisting of Arg, Ser, Gly and Glu.

Further, according to an embodiment of the present invention, the Xaa₅ is an amino acid selected from the group consisting of Asp, Glu, Arg, His, Lys, Ala, Val, Ile, Leu, Met, Phe, Tyr, Trp and Pro except for the polar amino acid. More preferably, the Xaa₅ is an amino acid selected from the group consisting of Pro, Glu, Lys, Ile and Met.

Further, according to an embodiment, the Xaa₆ and Xaa₇ are individually an amino acid selected from the group consisting Ala, Val, Ile, Leu, Met, Phe, Tyr and Trp as a hydrophobic amino acid and Ser, Thr, Asn and Gln as a polar amino acid. More preferably, the Xaa₆ is an amino acid selected from the group consisting of Thr, Asn, Gln, Met, Tyr and Trp. More preferably, the Xaa₇ is an amino acid selected from the group consisting of Ala, Met and Gln.

Further, according to an embodiment, the Xaa₅ is an amino acid of Lys, Ala or Arg. The Xaa₉ is an amino acid selected from the group consisting of Asp and Glu as an acidic amino acid and Ser, Thr, Asn and Gln as a polar amino acid. More preferably, the Xaa₉ is Asp, Glu, Asn or Gln.

According to an embodiment of the present invention, the peptide including the amino acid sequence represented by the Formula I is a polypeptide including a zinc finger domain of a retrovirus, in which the polypeptide may be selected from the group consisting of polypeptide having the amino acid sequences represented by SEQ ID NO: 17 to SEQ ID NO: 23.

Another aspect of the present invention provides an intracellular delivery carrier in which a cargo of an object to be delivered into a cell is conjugated with the end of an NC peptide of a retrovirus.

As used herein, the term “NC peptide” refers to a polypeptide that constitutes the nucleocapsid of a retrovirus. It is known that the polypeptide binds strongly to the retroviral genomic RNA to form the ribonucleoprotein core complex.

As used herein, the term “cell penetrating peptide (CPP)” refers to a peptide having the ability to carry a cargo of an object to be delivered into a cell in vitro and/or in vivo. The cell penetrating peptide means a peptide having an amino acid sequence that can pass through the cell membrane of a phospholipid bilayer itself.

According to an embodiment of the present invention, the retrovirus may be selected from the group consisting of human immunodeficiency virus (HIV), murine leukemia virus (MLU), simian immunodeficiency virus (SIV) and rous sarcoma virus (RSV) and may preferably be HIV.

As used herein, the term “cargo” refers to a chemical substance, a small molecule, a polypeptide, a nucleic acid and the like, which can be conjugated with NC peptides that act as a cell membrane penetrating peptide to be carried into cells.

In the present invention, the substance that can be conjugated with the end of the NC peptide of retrovirus or the peptide represented by the Formula I, that is, a substance that can be a cargo, may be various. For example, the substance includes a protein (polypeptide), a nucleic acid (polynucleotide), a chemical substance (drug), and the like, but is not limited thereto.

For example, it can be a drug, a contrast agent (e.g., T1 contrast agent, T2 contrast agent such as a superparamagnetic substance, a radioisotope, etc.), a fluorescent marker, a dyeing agent, and the like, but is not limited thereto. The polypeptide is a polymer of amino acids composed of two or more residues and includes peptides and proteins. Polypeptides may be, for example, proteins that are involved in cell immortalization (e.g., SV40 large T antigen and telomerase), anti-apoptotic proteins (e.g., mutant p53 and BclxL), antibodies, cancer genes (e.g., ras, myc, HPV E6/E7 and adenoviridae Ela), cell cycle regulating proteins (e.g., cyclin and cyclin-dependent phosphorylase) or enzymes (e.g., green fluorescent protein, beta-galactosidase and chloramphenicol acetyltransferase), but is not limited thereto. Also, the nucleic acid may be, for example, RNA, DNA or cDNA, and the sequence of the nucleic acid may be an encoding site sequence or a non-coding site sequence (e.g., an antisense oligonucleotide or a siRNA). Nucleotides as nucleic acid cargo may be standard nucleotides (e.g., adenosine, cytosine, guanine, thymine, inosine and uracil) or analogs (e.g., phosphorothioate nucleotides). For example, the nucleic acid cargo may be an antisense sequence consisting of a phosphorothioate nucleotide or RNAi.

According to an embodiment of the present invention, the substance conjugated with the NC peptide or the peptide represented by the Formula I of the present invention penetrates the cell membrane with very high efficiency and remains in the cytoplasm and the nucleus in the cell.

According to an embodiment of the present invention, the NC peptide may have one or more zinc finger domains and preferably two zinc finger domains. According to an embodiment, the NC peptide may be selected from the group consisting of polypeptides having the amino acid sequences represented by SEQ ID NO: 12 to SEQ ID NO: 15.

Another aspect of the present invention provides a method for delivering a cargo of an object to be delivered into a cell, the method including contacting the intracellular delivery carrier in which a cargo of an object to be delivered into a cell is conjugated with the end of a peptide including an amino acid sequence represented by the Formula I or an intracellular delivery carrier in which a cargo of an object to be delivered into a cell is conjugated with the end of an NC peptide of retrovirus with a cell.

Since the methods of the present invention utilize intracellular delivery carriers, the description common to both is omitted in order to avoid the excessive complexity of the present application.

Meanwhile, when the peptide containing the amino acid sequence represented by the Formula I to which a cargo is conjugated or the NC peptide to which a cargo is conjugated contacts the cell membrane in vitro or in vivo, the cargoes conjugated with peptides are delivered into the cell. There is no particular requirement for the contact between the intracellular delivery carrier and the cell membrane, for example, a limited time, temperature and concentration. It can be carried out under the general conditions applicable to cell membrane penetration in the art.

### Advantageous Effects

The intracellular delivery carrier including the peptide including the amino acid sequence represented by the Formula I of the present invention or the intracellular delivery carrier including the NC peptide has an advantage of efficiently transferring substances into cells even at a low concentration thereof compared with the existing peptide derived from the virus.

### BRIEF DESCRIPTION OF DRAWINGS

FIG. 1 shows the results of confirming the expression of NC-EGFP protein by SDS-PAGE.

FIGS. 2A and 2B show peptide sequences of several retroviruses (FIG. 2A) and the results of comparing the cell membrane penetration activity of several NC peptides conjugated with FITC, in which the box represents the zinc finger domain (FIG. 2B).

FIGS. 3A to 3C show the results of confirming the cell membrane penetration activity of FITC-HIV-NC peptide (nucleocapsid peptide) in RAW264.7 cells in a concentration-dependent manner (FIGS. 3A and 3B) or time-dependent manner (FIG. 3C).

FIG. 4 shows the result of confirming the cell membrane penetration activity of the HIV-NC peptide by immunostaining.

FIGS. 5A to 5E show the results of confirming the cell membrane penetration activity of FITC-HIV-NC peptides in RAW264.7 (FIG. 5A), THP-1 (FIG. 5B), CEM (FIG. 5C), MDCK (FIG. 5D) and 293FT (FIG. 5E) in a concentration-dependent manner.

FIGS. 6A to 6C show the results of comparing cell membrane penetration activity of GFP-HIV-NC peptide (FIG. 6A) and GFP-Tat peptide (FIG. 6B) in RAW264.7 cells in a concentration-dependent manner (FIG. 6C).

FIG. 7 shows the results of comparing cell membrane penetration activity of FITC-HIV-NC peptide, FITC-Tat peptide, FITC-MA11 peptide, FITC-PTD-ys peptide, FITC-TLM peptide and FITC-TD1 peptide.

FIG. 8 shows the result of confirming the degree of cell membrane penetration activity of FITC-13NC35 peptide.

FIG. 9 shows the results of confirming the degree of cell membrane penetration activity of FITC-29NC50 peptide.

FIG. 10 shows the results of confirming whether the distribution of HIV-NC peptides in tissue cells after the penetration into tissues in vivo in which the scale bar represents 50 μm in each photograph.

FIG. 11A and FIG. 11B show the results of confirming the distribution of HIV-NC peptides injected through the intravitreal (FIG. 11A) and subretinal (FIG. 11B) injection routes in retinal cells after the penetration into ocular tissue: GCL means ganglion cell layer, INL means inner nuclear layer, and ONL means outer nuclear layer.

FIG. 12 shows the results of exhibiting cell membrane penetration activity of hexapeptide conjugated with HIV-NC peptide.

FIG. 13A and FIG. 13B show the results of exhibiting the cell membrane penetration activity of RNA conjugated with HIV-NC peptide, specifically the GFP fluorescence signal was increased according to the treatment concentration of HIV-NC peptide (FIG. 13B), and the degree of cell membrane penetration activity of siGLO was increased (FIG. 13A).

### DETAILED DESCRIPTION OF THE INVENTION

Hereinafter, one or more embodiments are described in more detail by way of Examples. However, these Examples are intended to illustrate one or more embodiments, and the scope of the present invention is not limited to these Examples.

### Experimental Method

1. Construction of a Recombinant Vector for NC Peptide Expression

A recombinant vector was prepared as follows to confirm the expression of a recombinant protein conjugated with NC peptide and EGFP and to purify the protein. Polymerase chain reaction (PCR) was performed using a primer including restriction enzyme recognition sequences in order to add restriction enzyme recognition sequences at the N-terminus of the NC peptide gene to allow NdeI which is a restriction enzyme (New England Biolabs; NEB, USA) to act on and at the C-terminus of the NC peptide gene to allow BspEI to act on. The primer sequences (SEQ ID NO: 1 and SEQ ID NO: 2) used in PCR and the PCR conditions, respectively, are shown in Tables 1 and 2 below.

TABLE 1

| | SEQ ID NO. | Sequence |
|---|---|---|
| NC-NdeI | SEQ ID NO: 1 | CATATGCAGCGGGGAACTT |
| NC-BspEI | SEQ ID NO: 2 | TCCGGAGTTTGCCTGTCTC |

TABLE 2

| PCR reactants | | PCR cycle | | |
|---|---|---|---|---|
| dH₂O | 37.5 μl | 95° C. | 2 minutes | |
| dNTP (10X) | 4 μl | 95° C. | 1 minute | |
| F primer (10 μM) | 1 μl | 65° C. | 30 seconds | 30 times |
| R primer (10 μM) | 1 μl | 74° C. | 4 minutes | |
| NC peptide gene | 1 μl | 74° C. | 5 minutes | |
| DNA Taq polymerase | 0.5 μl | 4° C. | Unlimited | |
| buffer | 5 μl | | | |
| total | 50 μl | | | |

Next, in order to add restriction enzyme recognition sequences at the N-terminus of enhanced green fluorescent protein (EGFP) gene to allow BspEI to act on and at the C-terminus of the EGFP gene to allow HindIII to act on, PCR was carried out under the same conditions as in Table 2 as described above. However, the primers of SEQ ID NO: 3 and SEQ ID NO: 4 described in Table 3 below were used as the primers.

TABLE 3

| | SEQ ID NO. | Sequence |
|---|---|---|
| EGFP-BspEI-F | SEQ ID NO: 3 | TCCGGAGTGAGCAAGGGCGA |
| EGFP-HindIII-R | SEQ ID NO: 4 | AAGCTTCTTGTACACTCTCGT |

The NC peptide gene and the EGFP gene to which the restriction enzyme recognition sequence was added were reacted at 16° C. for 12 hours, resulting in the ligation. PCR was performed on the ligated NC peptide-EGFP gene (hereinafter referred to as ‘NC-EGFP gene’) (using primers of SEQ ID NOS: 1, 2, 3 and 4) to obtain the entire sequence of NC-EFGP gene. The conditions of NC-EGFP gene ligation reaction are shown in Table 4 below.

TABLE 4

| | |
|---|---|
| dH₂O | 6 μl |
| T4 DNA Ligase buffer (10X) | 1 μl |
| NC peptide DNA (50 ng/ [symbol] ) | 1 μl |
| EGFP gene DNA (50 ng/ [symbol] ) | 1 μl |
| T4 DNA Ligase (400 units/μl) | 1 μl |
| Total | 10 μl |

Then, pET21a (Novagen, USA) vector was cut with NdeI and HindIII restriction enzymes. Then, vector fragments were isolated according to the manufacturer's protocol using a PCR purification kit (Qiagen, USA). The restriction enzyme reaction was performed using NEBuffer #2 at 37° C. for 2 hours. The isolated pET21a vector fragment was ligated with the NC-EGFP gene, and then the recombination vector was isolated using the PCR Purification Kit. The recombinant vector into which the NC-EGFP gene was inserted was named pET21a NC-EGFP.

In order to replicate the pET21a NC-EGFP vector, the vector was transformed into E. coli DH5α and shake-cultured at 37° C. until the OD was 0.5 to 0.6 in LB liquid medium. After the culture was completed, the culture solution was centrifuged to collect the E. coli pellet, and the pET21a NC-EGFP vector was isolated from the E. coli pellet collected according to the manufacturer's protocol using a plasmid extraction kit (Qiagen). The isolated pET21a NC-EGFP vector was identified by quantifying the concentration using the UV method.

2. Expression, Isolation and Purification of NC-EGFP

In order to confirm the protein expression of the pET21a NC-EGFP vector prepared as described above, the following experiment was conducted.

PET21a NC-EGFP vector was transformed into BL21 (DE3) (Thermo Fisher, USA), plated on LB plate and cultured at 37° C. for 12 hours. Colonies formed after 12 hours were inoculated into LB liquid medium and further cultured at 37° C. After about 12 hours, the culture solution having reached an OD of 0.5 to 0.6 was inoculated into 250 ml of LB liquid medium and cultured at 37° C. for 3 hours to 4 hours to have an OD of 0.5 to 0.6. When the OD of the culture solution reached 0.5 to 0.6, 0.5 mM isopropyl β-D-thiogalactoside (IPTG) was added to the culture solution. Then, the culture was performed at 25° C. for 24 hours. After 24 hours, the culture solution was centrifuged to obtain BL21 pellet. The obtained pellet was suspended in a dissolution buffer (50 mM NaH₂PO₄, 300 mM NaCl, 10 mM imidazole and pH 8.0), and then the E. coli was disrupted (amplitude 40%) using an ultrasonic wave crusher.

The E. coli debris was centrifuged and separated into supernatant and precipitate, and the supernatant was filled into a tube using a 0.45 μm filter. The supernatant filled in the tube was placed in a column packed with Ni-NTA (nitrilotriacetic acid) resin to bind the protein and the resin to each other. In order to remove foreign proteins that did not bind to the resin, the resin was then washed with wash buffer (50 mM NaH₂PO₄, 300 mM NaCl, 20 mM imidazole and pH 8.0). The final protein was obtained in a gradient mobile phase using an imidazole-added buffer (50 mM NaH₂PO₄, 300 mM NaCl, 250 mM imidazole and pH 8.0). Next, in order to remove imidazole, the obtained protein was placed in a membrane tube, and buffer exchange by osmotic action was performed using a buffer (20 mM NaH₂PO₄, 300 mM NaCl and pH 7.2) not containing imidazole. Finally, the concentration of the protein dissolved in the buffer not containing imidazole was measured by Bradford assay to identify NC-EGFP protein expression. The results confirmed that even if other sequences were conjugated with the NC gene sequence, it did not affect the NC peptide expression.

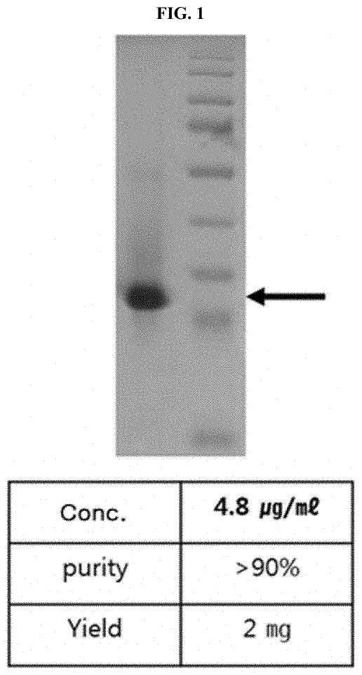

FIG. 1 shows the results of the NC-EGFP protein by SDS-PAGE, which shows that the NC-EGFP protein having a size of about 35 kDa was expressed.

3. Analysis of Cell Membrane Penetration Activity of NC Peptides

3-1. Cell Culture

HeLa, RAW 264.7 and 293 FT cells were cultured in DMEM medium supplemented with 10% FBS and 100 U/ml penicillin/streptomycin. MDCK cells were cultured in EMEM medium supplemented with 10% FBS and 100 U/ml penicillin/streptomycin (Hyclone, Logan, Utah, USA). THP-1 and CEM cells were cultured in RPMI-1640 medium supplemented with 10% FBS and 100 U/ml penicillin/streptomycin (Hyclone, Logan, Utah, USA). All cells were cultured in a humidified thermostat in 5% CO₂ at 37 C.

3-2. Analysis of Cell Membrane Penetration Activity of NC Peptide—Fluorescence-Activated Cell Sorting (FACS)

Various types of cells cultured in the Example 3-1 as described above were seeded into 6-well plates at a density of 1×10⁶ cells/well, and cultured for 24 hours to attach the cells to the plates. Then, cells were treated with the FITC-conjugated NC peptide (hereinafter referred to as “FITC-NC peptide”), the EGFP-conjugated NC peptide (hereinafter referred to as “NC-EGFP peptide”) and the EGFP-conjugated Tat peptide (hereinafter referred to as “Tat-EGFP peptide”), respectively, at different peptide concentrations (0.5, 1.0, 2.5, 5.0 and 10 μM) or different peptide treatment time. The peptides were prepared by chempeptide (China) and Life tein (USA). The cells were then washed three times with PBS, and the cells were separated with trypsin-EDTA and suspended in PBS containing 2% FBS/0.1% bovine serum albumin (BSA). The suspension was centrifuged at 2,000 rpm for 2 minutes to obtain cell pellets, and the cells obtained were fixed with 3.7% formaldehyde for 20 minutes. Then, cell pellets were obtained by centrifugation at 2,000 rpm for 2 minutes, washed twice with PBS and then suspended in PBS containing 2% FBS and 0.1% BSA. Cells were analyzed by flow cytometry using a fluorescence activated cell sorter (FACS Calibur, Beckon Dickinson, Calif., USA).

3-3. Analysis of Cell Membrane Penetration Activity of NC Peptide—Immunostaining

HeLa cells were seeded into a 12-well plate having glass at a density of 1×10⁵ cells/well, and then cultured for 24 hours to attach the cells to the glass. Then, cells were treated with FITC-NC peptides at 3 mM concentration for 3 hours. After 3 hours, the cells were washed three times with PBS, the cells were fixed with 3.7% formaldehyde for 20 minutes, and the cells were treated with PBS containing 0.2% Triton X-100 to increase the cell membrane penetration activity. Then, the cells were blocked with 3% BSA for 1 hour, reacted with lamin A/C antibody (Sigma-Aldrich, USA) at room temperature for 2 hours, and washed three times with PBS. Next, the cells were treated with Cy3-conjugated secondary antibody (Jackson ImmunoResearch, USA) at room temperature for 1 hour, washed twice with PBS, and then stained with DAPI (4′,6-diamidino-2-phenylindol) for 10 minutes. The HeLa cell-attached glass was removed and placed on a slide glass. Then, the cells were observed with a confocal laser scanning microscope (LSM 700, Zeiss, Germany).

In addition, the following method was used to compare the penetration activity of NC peptides with other cell penetrating peptides.

The HeLa cells were seeded into a 12-well plate containing glass at a density of 1×10⁵ cells/well, and then cultured for 24 hours to attach the cells to the glass. Thereafter, the FITC-NC peptide, the trans-activating transcriptional activator peptide (FITC-Tat peptide), the FITC-MA11 peptide, the protein transduction domain-ys peptide (FITC-PTD-ys peptide), the translocation motif peptide (FITC-TLM peptide) and FITC-TD1 peptide were treated with the HeLa cells for 3 hours. After 3 hours, the cells were washed 3 times with PBS and the cells were fixed with 3.7% formaldehyde for 20 minutes. The cells were treated with PBS containing 0.2% Triton X-100 to increase cell membrane penetration activity and blocked with 3% BSA for 1 hour. Then, the cells were reacted with tubulin antibody (Santa Cruz Biotechnology, USA) at room temperature for 2 hours and washed three times with PBS. The cells were treated with Cy3 secondary antibody. They were reacted at room temperature for 1 hour, washed twice with PBS, and stained with DAPI for 10 minutes. The HeLa cell-attached glass was removed and placed on a slide glass. Then, the cells were observed with a confocal laser scanning microscope.

Further, in order to identify a change in cell membrane penetration activity by FITC-NC peptide, the HeLa cells were treated with hexapeptide, FITC-hexapeptide, FITC-NC-hexapeptide, FITC-13NC35 peptide and FITC-29NC50 peptide for 3 hours in the same manner as above and observed with a confocal laser scanning microscope. Meanwhile, the amino acid sequences of the hexapeptide, PTD-ys peptide, TLM peptide, TD1 peptide, NC-hexapeptide, 13NC35 peptide and 29NC50 peptide used are shown in Table 5 below.

TABLE 5

| Name | SEQ ID NO. | Amino acid Sequence |
|---|---|---|
| Hexa peptide | SEQ ID NO: 5 | EEMQRR |
| PTD-ys | SEQ ID NO: 6 | YARVRRRGPRR |
| peptide | | |
| TLM peptide | SEQ ID NO: 7 | PLSSIFSRIGDP |
| TD1 peptide | SEQ ID NO: 8 | KAMININKFLNQC |
| NC-hexa | SEQ ID NO: 9 | VKCFNCGKEGHTARNCRAPRKKG |
| peptide | | CWKCGKEGHQMKDCTEEEMQRR |
| 13NC35 | SEQ ID NO: 10 | VKCFNCGKEGHTARNCRAPRKKG |
| peptide | | |
| 29NC50 | SEQ ID NO: 11 | RAPRKKGCWKCGKEGHQMKDCT |
| peptide | | |

3-4. Analysis of Cell Membrane Penetration Activity of NC Peptide—Fluorometer

RAW264.7 cells were seeded into 24-well plates at a density of 5×10⁴ cells/well and cultured for 24 hours to attach the cells to the plates. Then, each of the Tat-EGFP peptide and the NC-EGFP peptide was treated at different concentrations for 1 hour. After 1 hour, the cells were washed three times with PBS, and 100 ml of radio-immunoprecipitation assay (RIPA) buffer was added to dissolve the cells at 4° C. for 30 minutes. 100 ml of the cell lysate was transferred to a 96-well plate for fluorescence analysis, and GFP fluorescence was measured using a fluorescence analyzer (Synergy MX, BIOTEK, USA).

Further, NC peptides of FITC-conjugated Human immunodeficiency virus (HIV), Murine leukemia virus (MLU), Simian immunodeficiency virus (SIV) and RSV (Rous sarcoma virus) (hereinafter referred to as FITC-HIV-NC, FITC-MLUNC, FITC—SIV—NC and FITC—RSV-NC, respectively) and Tat peptide were treated with RAW264.7 cells at a concentration of 1.0 μM for 1 hour. Then, the fluorescence signal of the cells was measured.

3-5. Change in RNA Transduction Efficiency by NC Peptide

MT4 cells were seeded into 24-well plates at a density of 2×10⁵ cells/well and cultured in a humidified thermostat in 5% CO₂ at 37° C. for 24 hours. 2 μl of NC peptides and 40 nM siGLO (Green transfection indicator, Dharmacon, D-001630-01-05) having different concentrations were mixed and reacted at room temperature for 30 minutes and then treated to MT4 cells. After further culture for 24 hours, MT4 cells were washed three times with PBS and analyzed by flow cytometry using a fluorescence activated cell sorter. Further, the cells were observed with a fluorescence inverted microscope (Olympus).

4. In Vivo Tissue Penetration of NC Peptide and Distribution of NC Peptide in Tissue Cells

The FITC-HIV-NC peptide and FITC-Tat peptide having a concentration of 3.2 mg/100 μl, respectively, were injected into mouse tail vein using a 30 gauge syringe. The kidney, liver, lung, heart and spleen of the mice were removed 2 hours after the injection of the peptide and fixed with 4% paraformaldehyde for 1 hour. They were placed in 30% sucrose until the tissue was subsided. Then, each tissue was frozen using an OCT compound (Leica, Germany), and a tissue section slide was prepared with a freezing sectional device. Tissue section slides were stained with DAPI for 10 minutes according to methods known in the art and observed with a confocal laser scanning microscope.

In addition, 1 μl of a 100 μM stock of FITC-HIV-NC peptide was intravitreally injected, and 2 μl of a 100 μM stock of FITC-HIV-NC peptide was subretinally injected. After 24 hours, mouse eye tissue was extracted. Eye tissue section slides were prepared in the same manner as described above. Slides were stained with DAPI for 10 min and observed with a confocal laser scanning microscope.

Experiment Result

1. Identification of Cell Membrane Penetration Activity of Various Retroviral NC Peptides

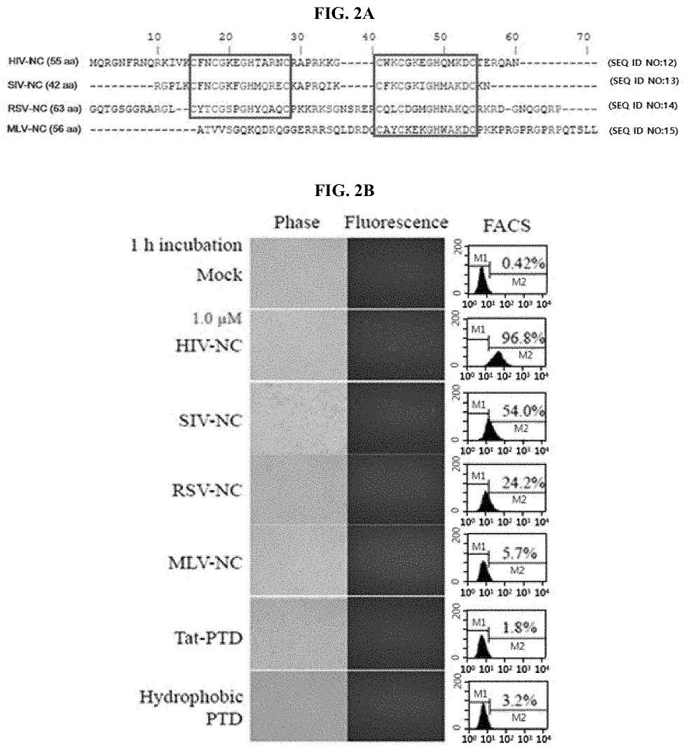

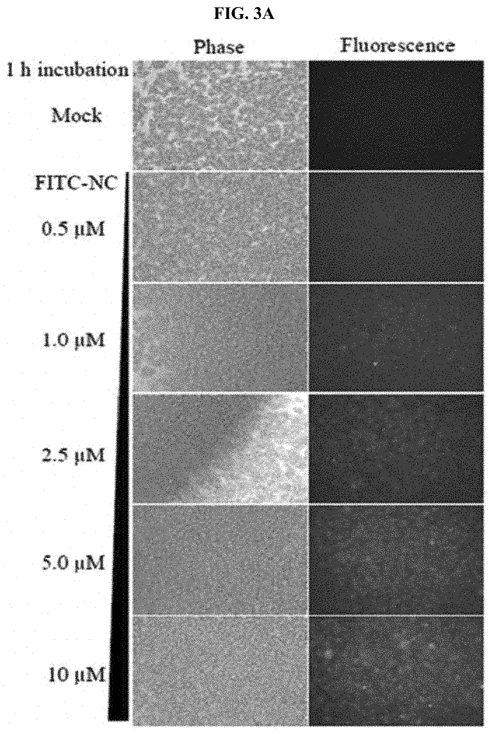

In order to confirm whether the degree of cell membrane penetration activity was varied depending on the degree of similarity of NC peptide sequence, RAW264.7 Cells were treated with 1.0 μM FITC-conjugated NC peptide of HIV, SIV, RSV and MLV and Tat peptide for 1 hour. As a result, as shown in FIG. 2, it was confirmed that NC peptides of HIV, SIV, RSV and MLV have excellent cell membrane penetration activity through the FITC fluorescence brightness. FACS analysis was performed to indicate that NC peptides of HIV, SIV, RSV and MLV, respectively, showed fluorescence in 96.8%, 54.0%, 24.2% and 5.7% of cells. The results of this experiment indicate that NC peptides of several retroviruses including HIV have excellent cell membrane penetration activity compared with that of the conventional Tat peptides. As shown in FIG. 2, it was confirmed that the NC peptides of HIV, SIV, RSV and MLV contained a zinc finger domain as a consensus sequence.

Meanwhile, the amino acid sequences of the NC peptides of HIV, SIV, RSV and MLV used above are shown in Table 6 below.

TABLE 6

| Name | SEQ ID NO. | Amino acid Sequence |
|---|---|---|
| HIV-NC | SEQ ID NO: 12 | MQRGNFRNQRKIVKCFNCGKEGHTARNC |
| peptide | | RAPRKKGCWKCGKEGHQMKDCTERQAN |
| SIV-NC | SEQ ID NO: 13 | RGPLKCFNCGKFGHMQRECKAPRQIKCF |
| peptide | | KCGKIGHMAKDCKN |
| RSV-NC | SEQ ID NO: 14 | GQTGSGGRARGLCYTCGSPGHYQAQCPK |
| peptide | | KRKSGNSRERCQLCDGMGHNAKQCRKRD |
| | | GNQGQRP |
| MLV-NC | SEQ ID NO: 15 | ATVVSGQKQDRQGGERRRSQLDRDQCAY |
| peptide | | CKEKGHWAKDCPKKPRGPRGPRPQTSLL |

2. Identification of Cell Membrane Penetration Activity of HIV-NC Peptide

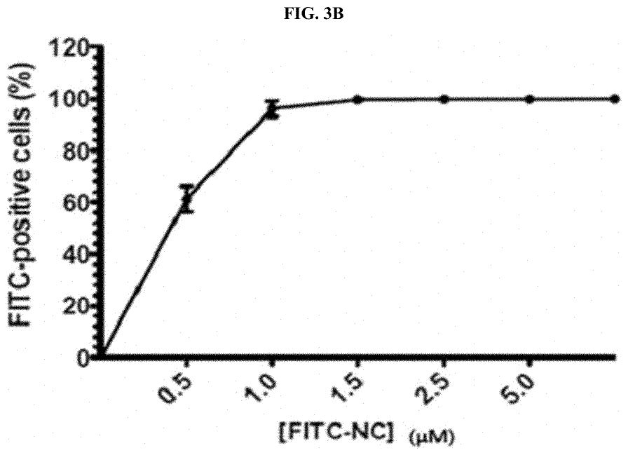

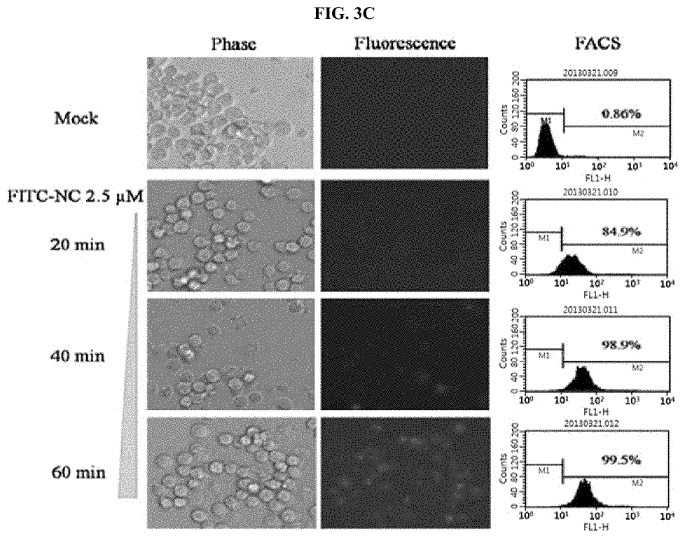

In order to examine the cell membrane penetration activity of HIV-NC peptide (SEQ ID NO: 12) having the highest cell membrane penetration activity in the above experiment according to its concentrations, RAW264.7 macrophages were treated with FITC-HIV-NC peptide at a concentration of 0.5, 1, 2.5, 5.0 and 10 μM for 1 hour. The fluorescence intensity of the cells was measured using a fluorescence microscope (Leica Micoscope Systems, Germany). As shown in FIG. 3A, it was confirmed that as the concentration of FITC-HIV-NC peptide increases, the penetration activity of the peptide increases. Further, as a result of the fluorescence analysis, as shown in FIG. 3B, it was confirmed that as the concentration of FITC-HIV-NC peptide was increased, the fluorescence signal was increased, indicating that the cell membrane penetration activity was increased.

Further, in order to confirm the degree of cell membrane penetration of FITC-HIV-NC peptides according to the treatment time, the peptides were treated with RAW 264.7 cells at a concentration of 2.5 μm for 20 minutes, 40 minutes and 60 minutes. As a result, it was confirmed by fluorescence microscopy and FACS analysis that as shown in FIG. 3C, the cell membrane permeability of the peptide was significantly increased with increasing time of FITC—HIV-NC peptide treatment.

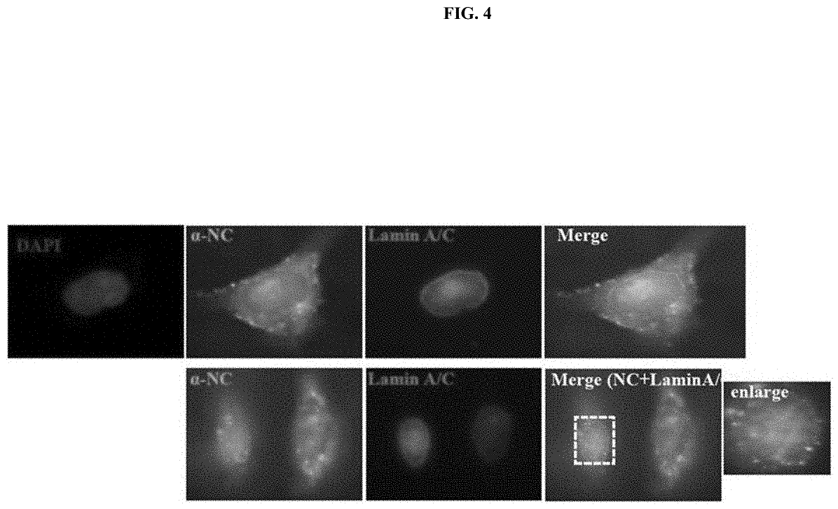

In order to verify the cell membrane penetration activity of HIV-NC peptides, HeLa cells were immunostained and observed with a confocal laser scanning microscope. As a result, it was confirmed that as shown in FIG. 4, many HIV-NC peptides were uniformly distributed in the cell membrane, cytoplasm and nuclear membrane and that some HIV-NC peptides were distributed in the nucleus. The results indicate that HIV-NC peptides can penetrate cell membranes at low concentrations and that HIV-NC peptides penetrate cell membrane quickly.

3. Identification of Cell Membrane Penetration Activity of HIV-NC Peptides in Various Cells

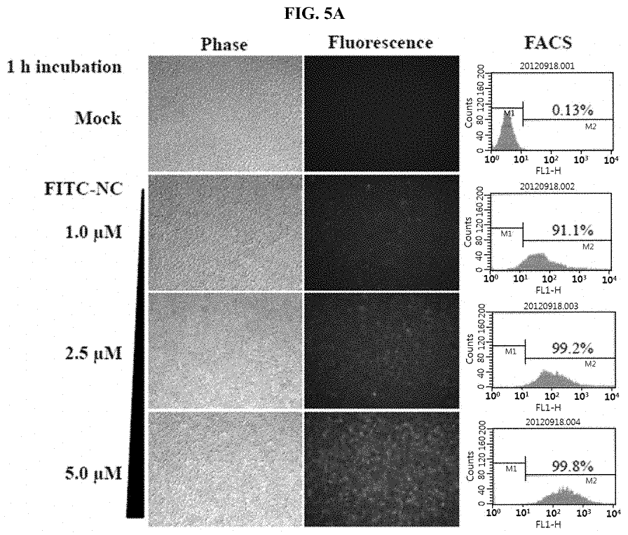

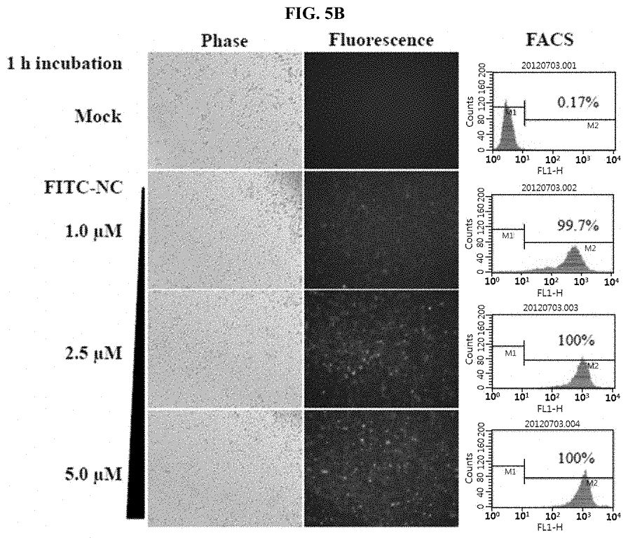

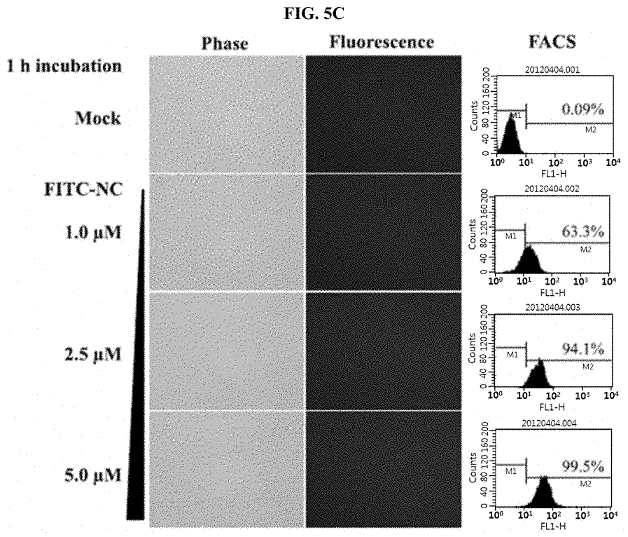

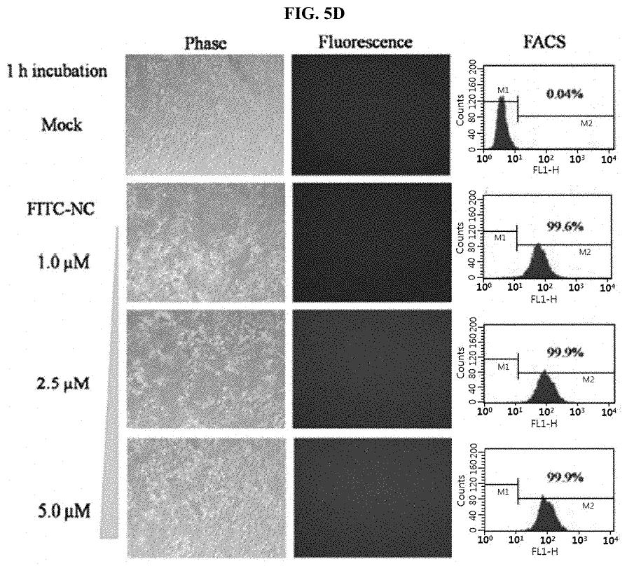

In order to identify whether the cell membrane penetration activity of HIV-NC peptide varies depending on kinds of cells, RAW264.7, THP-1, CEM, MDCK and 293FT cells were treated with FITC-HIV-NC peptide at a concentration of 1.0, 2.5 and 5.0 μM for 1 hour or 2 hours. As a result, it was confirmed by fluorescence microscopy and FACS analysis that as shown in FIGS. 5A to 5E, the cell membrane penetration activity of the peptide was significantly increased as the concentration of FITC-HIV-NC peptide was increased. FIG. 5A shows the results of RAW264.7 (mouse macrophage). FIG. 5B shows the results of THP-1 (human mononuclear cell line) cells, FIG. 5C shows the results of CEM. FIG. 5D shows the results of MDCK (Madin-Darby canine kidney), and FIG. 5E shows the results of 293FT (derived from human fetal kidney cells). The above results indicate that HIV-NC peptides can penetrate various cell membranes of cancer cells, immune cells, epithelial cells and the like.

4. Comparison of Cell Membrane Penetration Activity Between HIV-NC and Tat Peptides

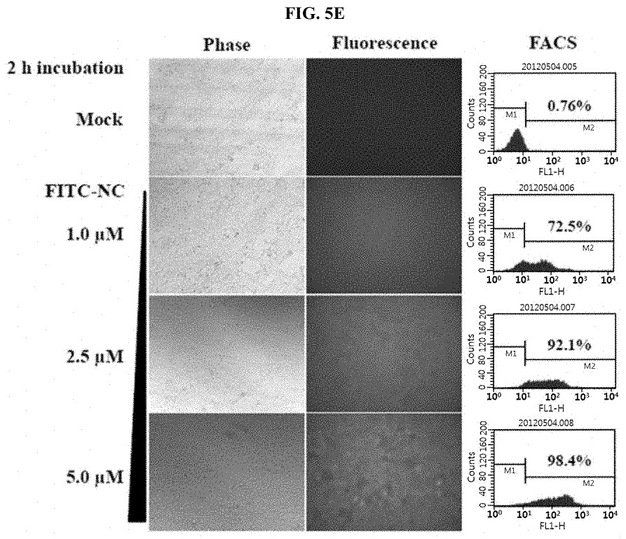

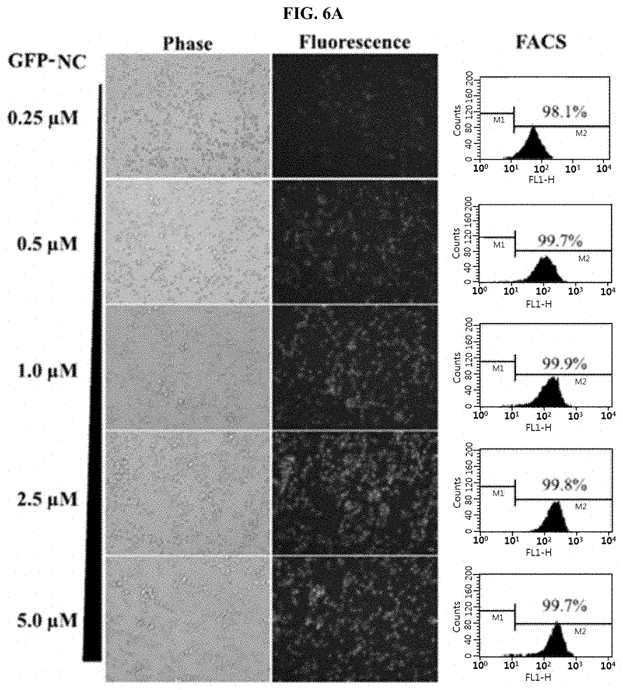

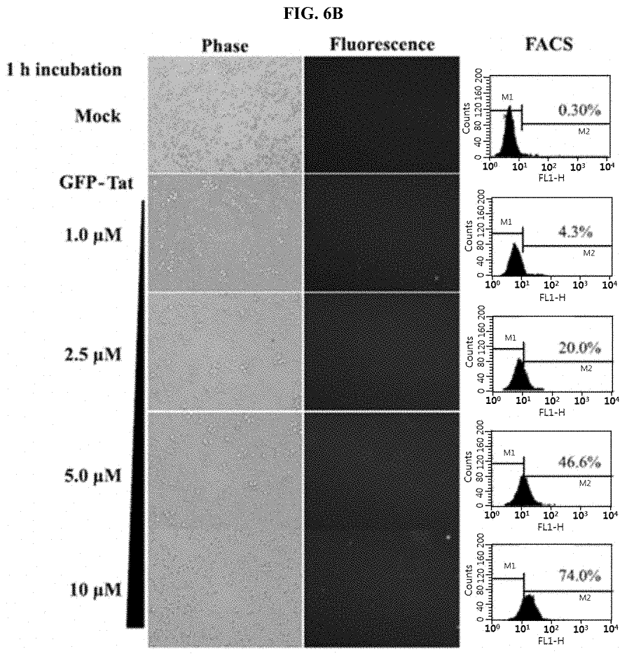

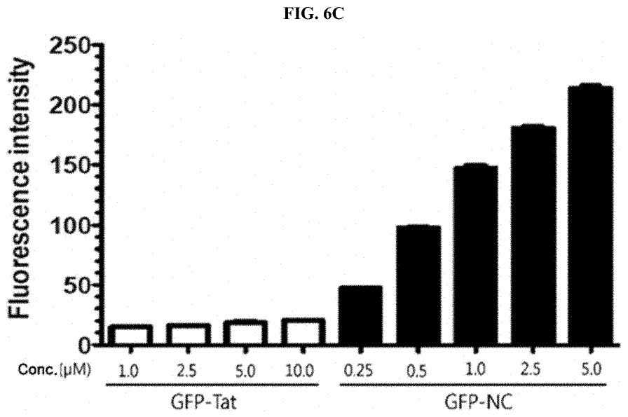

In order to confirm the degree of cell membrane penetration activity of HIV-NC peptides, a comparative experiment carried out with a Tat peptide known as a conventional cell penetrating protein. RAW264.7 cells were treated with green fluorescent protein (GFP)-conjugated HIV-NC peptide (hereinafter referred to as GFP-HIV-NC peptide) at 0.5, 1.0, 2.5 and 5.0 μM and GFP-conjugated Tat peptide at 1.0, 2.5, 5.0 and 10 μM, respectively, for 1 hour. After 1 hour, the fluorescence of the peptides was confirmed. As a result, it was confirmed that as shown in FIGS. 6A and 6B, the GFP-HIV-NC peptide showed fluorescence at a low concentration of 0.25 μM, while the GFP-Tat peptide showed slightly fluorescence at a high concentration of 10 μM. Further, the cells were collected and analyzed by FACS. As a result, it was confirmed that as shown in FIGS. 6A and 6B, GFP-HIV-NC peptides showed fluorescence for 99.9% of cells at a concentration of 1 μM, while GFP-Tat peptides showed fluorescence for 4.3% of cells at the same concentration.

Further, as a result of the fluorescence analysis, it was confirmed that as shown in FIG. 6C, as the concentration of GFP-HIV-NC peptide was increased, the cell membrane penetration activity was also increased, but the GFP-Tat peptide has very low cell membrane penetration activity regardless of the treatment concentration. The results indicate that HIV-NC peptides are highly efficient cell penetrating peptides as compared with Tat peptides, which are well-known as an existing cell penetrating protein.

5. Comparison of Cell Membrane Penetration Activity of HIV-NC Peptides and Other Cell Penetrating Peptides

The HeLa cells were treated with FITC-HIV-NC peptides, FITC-Tat peptides, FITC-MA11 peptides, FITC-PTD-ys peptides, FITC-TLM peptides and FITC-TD1 peptides, and then the penetration activity of each peptide was confirmed.

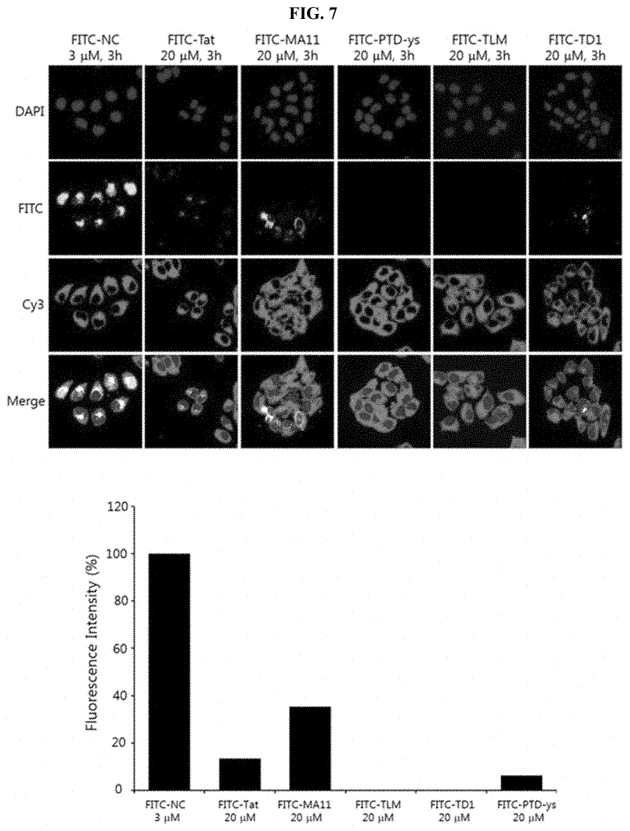

As a result, it was confirmed that as shown in FIG. 7, the FITC-TLM peptides and the FITC-TD1 peptides were hardly penetrable to the cell membrane even though they were treated at a high concentration of 20 μM, but the fluorescence signal was strong and the cell membrane penetration activity was remarkably excellent although FITC-HIV-NC peptides were treated at a low concentration of 3 μM. Further, the cell membrane penetration activity of FITC-HIV-NC peptides is about 2.5 times higher than that of FITC-MA11 peptides, about 7 times higher than that of FITC-Tat peptides and about 10 times higher than that of FITC-PTD-ys peptides.

6. Identification of the Cell Membrane Penetration Activity of Zinc Finger Domains in NC Peptides

Experimental result 1 confirmed that several retroviral NC peptides had cell membrane penetration activity. As shown in FIG. 2, it was confirmed that there are two zinc finger domains in HIV, SIV and RSV, and one zinc finger domain in MLV.

In order to confirm whether the cell membrane penetration activity is caused by the zinc finger domain which is a consensus sequence in several NC peptides, FITC was conjugated with a peptide containing the first zinc finger domain consisting of the 13th amino acid to the 35th amino acid of the HIV-NC peptide (13NC35 peptide, SEQ ID NO: 10), and, FITC was conjugated with a peptide containing the second zinc finger domain consisting of the 29th amino acid to the 50th amino acid of the HIV-NC peptide (29NC50 peptide, SEQ ID NO: 11) so that the cell membrane penetration activity was confirmed in HeLa cells, respectively.

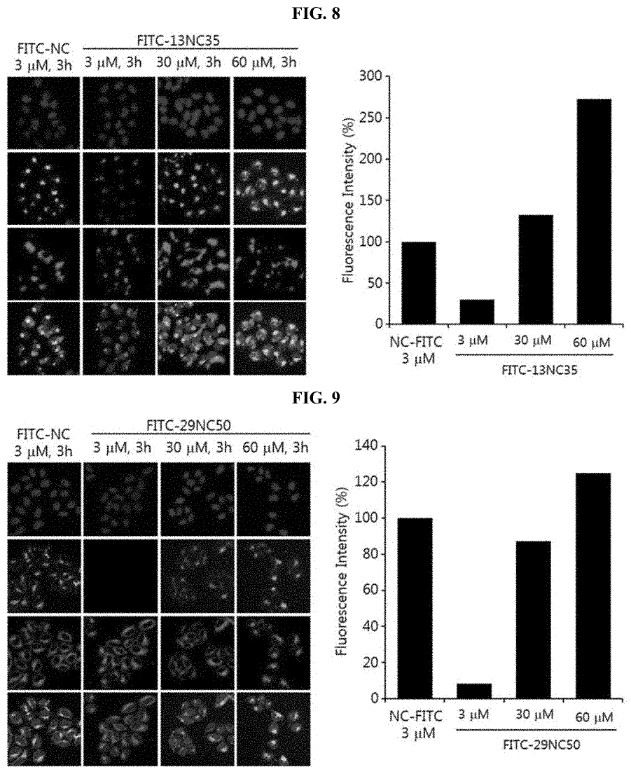

As a result, it was confirmed that as shown in FIGS. 8 and 9, fluorescence signals were increased in proportion to the concentration of both 13NC35 peptide and 29NC50 peptide. This means that the zinc finger domain of the NC peptide is an important part of cell membrane penetration activity.

Meanwhile, the amino acid sequences of the zinc finger domains of NC peptides of HIV, SIV, RSV and MLV are shown in Table 7 below.

TABLE 7

| Name | SEQ ID NO. | Amino acid Sequence |
|---|---|---|
| HIV-NC 1^{st} Zinc | SEQ ID NO: 17 | CFNCGKEGHTARNC |
| finger domain | | |
| HIV-NC 2^{nd} Zinc | SEQ ID NO: 18 | CWKCGKEGHQMKDC |
| finger domain | | |
| SIV-NC 1^{st} Zinc | SEQ ID NO: 19 | CFNCGKEGHMQREC |
| finger domain | | |
| SIV-NC 2^{nd} Zinc | SEQ ID NO: 20 | CFKCGKIGHMAKDC |
| finger domain | | |
| RSV-NC 1^{st} Zinc | SEQ ID NO: 21 | CYTCGSPGHYQAQC |
| finger domain | | |
| RSV-NC 2^{nd} Zinc | SEQ ID NO: 22 | CQLCDGMGHNAKQC |
| finger domain | | |
| MLV-NC Zinc | SEQ ID NO: 23 | CAYCKEKGHWAKDC |
| finger domain | | |

7. In Vivo Tissue Penetration of HIV-NC Peptide and its Distribution in Tissue Cells

In order to confirm whether the NC peptides penetrate into tissues in vivo and are uniformly distributed in tissue cells, HIV-NC peptides were injected to the mouse tail vein. As a result, it was confirmed that as shown in FIG. 10, the peptides were well distributed in tissue cells of kidney, liver, lung, heart and spleen. In particular, the distribution efficiency to the kidney, liver and lung tissue cells was remarkably excellent. On the other hand, it was confirmed that Tat peptides were hardly distributed in other tissues except for kidney.

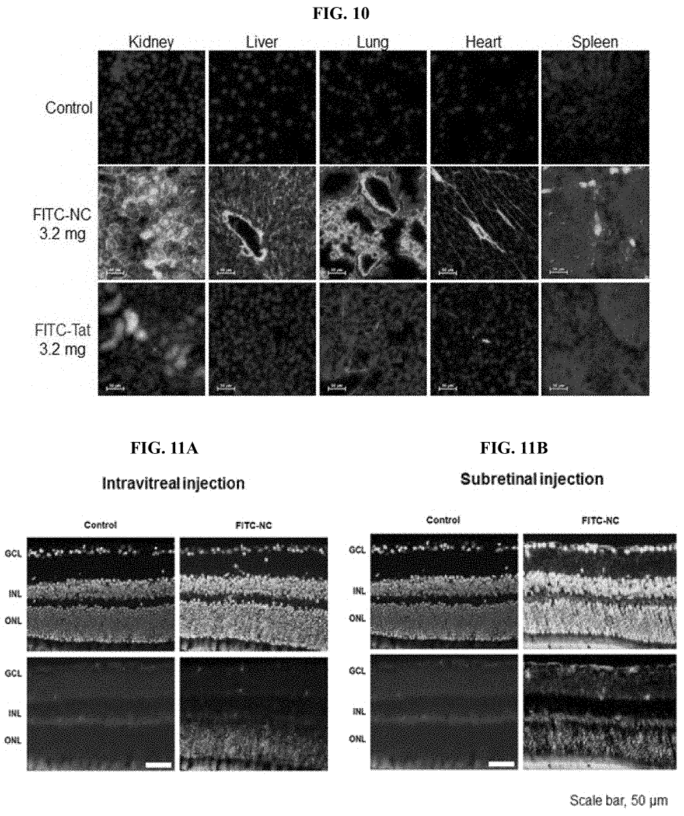

Further, HIV-NC peptides were injected through the intravitreal and subretinal injection routes. As a result, it was confirmed that as shown in FIG. 11, the fluorescence signal was strong so that HIV-NC peptides penetrated the eye tissue through the eye injection route to be well distributed in retinal cells.

8. Confirmation of Cell Membrane Penetration Activity of Polypeptide by HIV-NC Peptide

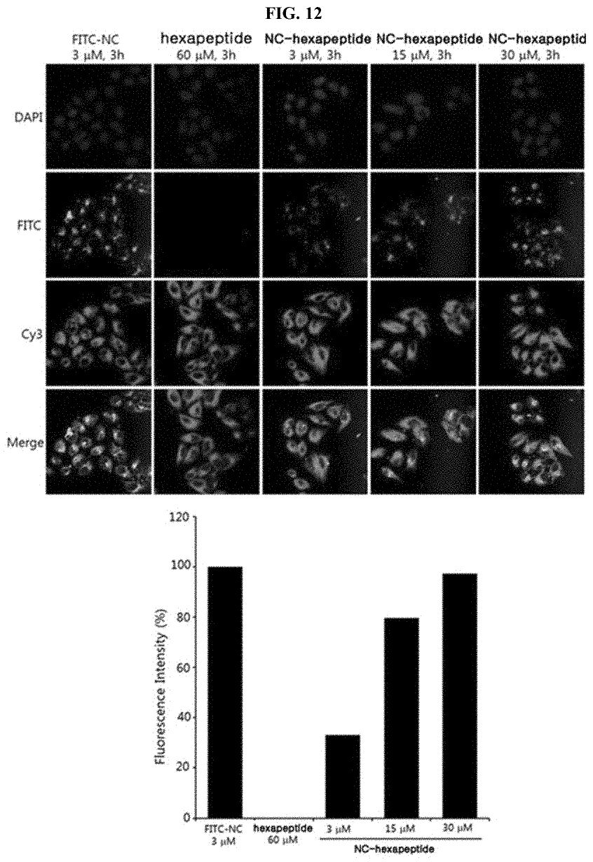

In order to confirm whether HIV-NC peptides using a polypeptide as a cargo can have cell membrane penetration activity, hexapeptide was conjugated to HIV-NC peptide, and cells were treated with the peptides to observe the fluorescence signal. As a result, it was confirmed that as shown in FIG. 12, the hexapeptide itself did not penetrate the cell membrane, but when conjugated with the HIV-NC peptide, they could penetrate cell membrane and that cell membrane penetration activity was increased in proportion to the concentration.

9. Confirmation of Cell Membrane Penetration Activity of Polynucleotide by HIV-NC Peptide

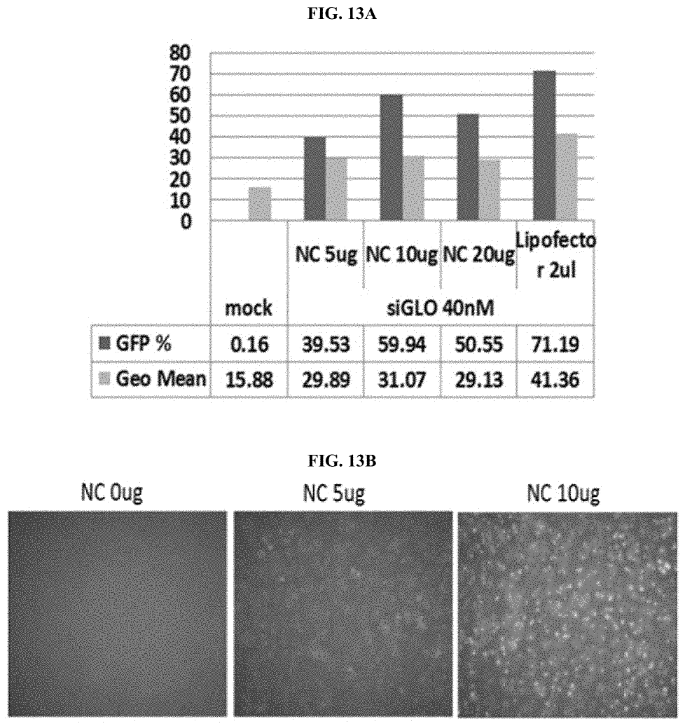

In order to confirm whether HIV-NC peptides using a polynucleotide as a cargo can have cell membrane penetration activity, siGLO RNA was conjugated to HIV-NC peptide, and cells were treated with the peptides to observe the fluorescence signal. As a result, it was confirmed that as shown in FIG. 13, the GFP fluorescence signal was increased according to the treatment concentration of HIV-NC peptide, and the degree of cell membrane penetration activity of siGLO was increased. These results indicate that HIV-NC peptides can significantly improve the cell membrane penetration activity of polynucleotides.

Hereinabove, the present invention has been described with reference to the preferred embodiments. It will be understood by those skilled in the art that the present invention may be carried out as modified embodiments without departing from the spirit and scope of the present invention. Therefore, the disclosed embodiments should be considered in an illustrative rather than a restrictive sense. It should be construed that the scope of the present invention is defined by the appended claims rather than by the foregoing description, and all differences within the scope of equivalents thereof should be included in the present invention.

1. A cell penetrating peptide comprising an amino acid sequence represented by the following Formula I:
[Formula I]
(SEQ ID NO: 16)
Cys-Xaa₁-Xaa₂-Cys-Xaa₃-Xaa₄-Xaa₅-Gly-His-Xaa₆-Xaa₇-
Xaa₈-Xaa₉-Cys
wherein Xaa1 is an amino acid selected from the group consisting of Ala, Val, Ile, Leu, Met, Phe, Tyr, Trp, Ser, Thr, Asn and Gln,
wherein Xaa2 is an amino acid selected from the group consisting of Ala, Val, Ile, Leu, Met, Phe, Tyr, Trp, Arg, His, Lys, Asn, Ser, Thr and Gln,
wherein Xaa3 and Xaa4 are individually an amino acid selected from the group consisting of Asp, Glu, Arg, His, Lys, Ser, Thr, Asn, Gln and Gly,
wherein Xaa5 is an amino acid selected from the group consisting of Asp, Glu, Arg, His, Lys, Ala, Val, Ile, Leu, Met, Phe, Tyr, Trp and Pro,
wherein Xaa6 and Xaa7 are individually an amino acid selected from the group consisting of Ser, Thr, Asn, Gln, Ala, Val, Ile, Leu, Met, Phe, Tyr and Trp,
wherein Xaa8 is an amino acid of Lys, Ala or Arg, and
wherein Xaa9 is an amino acid selected from the group consisting of Asp, Glu, Ser, Thr, Asn and Gln.

2. An intracellular delivery carrier in which a cargo of an object to be delivered into a cell is conjugated with the end of a cell penetrating peptide of claim 1.

3. The intracellular delivery carrier of claim 2, wherein the cargo of the object to be delivered into a cell is a chemical substance, a polypeptide, a nucleic acid, a carbohydrate or a lipid.

4. The intracellular delivery carrier of claim 2, wherein the peptide including the amino acid sequence represented by the 2A I is selected from the group consisting of polypeptides having the amino acid sequence represented by SEQ ID NO: 17 to SEQ ID NO: 23.


SEQUENCE LISTING (23 sequences)

SEQ ID NO: 1 - DNA, 19 bp, Artificial Sequence
  synthetic polynucleotide
catatgcagcggggaactt

SEQ ID NO: 2 - DNA, 19 bp, Artificial Sequence
  synthetic polynucleotide
tccggagtttgcctgtctc

SEQ ID NO: 3 - DNA, 20 bp, Artificial Sequence
  synthetic polynucleotide
tccggagtgagcaagggcga

SEQ ID NO: 4 - DNA, 21 bp, Artificial Sequence
  synthetic polynucleotide
aagcttcttgtacactctcgt

SEQ ID NO: 5 - protein, 6 aa, Artificial Sequence
  hexapeptide
EEMQRR

SEQ ID NO: 6 - protein, 11 aa, Artificial Sequence
  PTD-ys peptide
YARVRRRGPRR

SEQ ID NO: 7 - protein, 12 aa, Artificial Sequence
  TLM peptide
PLSSIFSRIGDP

SEQ ID NO: 8 - protein, 13 aa, Artificial Sequence
  TD1 peptide
KAMININKFLNQC

SEQ ID NO: 9 - protein, 45 aa, Artificial Sequence
  NC-hexapeptide
VKCFNCGKEGHTARNCRAPRKKGCWKCGKEGHQMKDCTEEEMQRR

SEQ ID NO: 10 - protein, 23 aa, Artificial Sequence
  13NC35 peptide
VKCFNCGKEGHTARNCRAPRKKG

SEQ ID NO: 11 - protein, 22 aa, Artificial Sequence
  29NC50 peptide
RAPRKKGCWKCGKEGHQMKDCT

SEQ ID NO: 12 - protein, 55 aa, Artificial Sequence
  HIV-NC peptide
MQRGNFRNQRKIVKCFNCGKEGHTARNCRAPRKKGCWKCGKEGHQMKDCTERQAN

SEQ ID NO: 13 - protein, 42 aa, Artificial Sequence
  SIV-NC peptide
RGPLKCFNCGKFGHMQRECKAPRQIKCFKCGKIGHMAKDCKN

SEQ ID NO: 14 - protein, 63 aa, Artificial Sequence
  RSV-NC peptide
GQTGSGGRARGLCYTCGSPGHYQAQCPKKRKSGNSRERCQLCDGMGHNAKQCRKRDGNQGQRP

SEQ ID NO: 15 - protein, 56 aa, Artificial Sequence
  MLV-NC peptide
ATVVSGQKQDRQGGERRRSQLDRDQCAYCKEKGHWAKDCPKKPRGPRGPRPQTSLL

SEQ ID NO: 16 - protein, 14 aa, Artificial Sequence
  Synthetic Polypeptide Formula I
  Xaa (2)..(2): Ala, Val, Ile, Leu, Met, Phe, Tyr, Trp, Ser, Thr, Asn or Gln
  Xaa (3)..(3): Ala, Val, Ile, Leu, Met, Phe, Tyr, Trp, Arg, His, Lys, Asn, Ser, Thr or Gln
  Xaa (5)..(5): Asp, Glu, Arg, His, Lys, Ser, Thr, Asn, Gln or Gly,
  Xaa (6)..(6): Asp, Glu, Arg, His, Lys, Ser, Thr, Asn, Gln or Gly,
  Xaa (7)..(7): Asp, Glu, Arg, His, Lys, Ala, Val, Ile, Leu, Met, Phe, Tyr, Trp or Pro
  Xaa (10)..(10): Ser, Thr, Asn, Gln, Ala, Val, Ile, Leu, Met, Phe, Tyr or Trp
  Xaa (11)..(11): Ser, Thr, Asn, Gln, Ala, Val, Ile, Leu, Met, Phe, Tyr or Trp
  Xaa (12)..(12): Lys, Ala or Arg
  Xaa (13)..(13): Asp, Glu, Ser, Thr, Asn or  Gln
CXXCXXXGHXXXXC

SEQ ID NO: 17 - protein, 14 aa, Artificial Sequence
  HIV-NC 1st zinc finger domain
CFNCGKEGHTARNC

SEQ ID NO: 18 - protein, 14 aa, Artificial Sequence
  HIV-NC 2nd zinc finger domain
CWKCGKEGHQMKDC

SEQ ID NO: 19 - protein, 14 aa, Artificial Sequence
  SIV-NC 1st zinc finger domain
CFNCGKEGHMQREC

SEQ ID NO: 20 - protein, 14 aa, Artificial Sequence
  SIV-NC 2nd zinc finger domain
CFKCGKIGHMAKDC

SEQ ID NO: 21 - protein, 14 aa, Artificial Sequence
  RSV-NC 1st zinc finger domain
CYTCGSPGHYQAQC

SEQ ID NO: 22 - protein, 14 aa, Artificial Sequence
  RSV-NC 2nd zinc finger domain
CQLCDGMGHNAKQC

SEQ ID NO: 23 - protein, 14 aa, Artificial Sequence
  MLV-NC zinc finger domain
CAYCKEKGHWAKDC